(19) United States
(12) Patent Application Publication
(10) Pub. No.: US 2015/0202021 A1
(43) Pub. Date: Jul. 23, 2015

(54) METHOD FOR PROCESSING IMAGES OF INTERVENTIONAL RADIOLOGY

(71) Applicant: GENERAL ELECTRIC COMPANY, Schenectady, NY (US)

(72) Inventors: Yves Trousset, Palaiseau (FR); Jeremie Pescatore, Le Chesnay (FR); Sebastien Gorges, Buc (FR); Vincent Bismuth, Buc (FR); Maria Carolina Vanegas Orozo, Paris (FR)

(21) Appl. No.: 14/556,966

(22) Filed: Dec. 1, 2014

Related U.S. Application Data
(63) Continuation of application No. 12/350,585, filed on Jan. 8, 2009, now abandoned.

(30) Foreign Application Priority Data
Jan. 10, 2008 (FR) 0850133

(51) Int. Cl. A61B 19/00

(52) U.S. Cl. CPC A61B 19/5244; A61B 2019/5265; A61B 2019/5295; A61B 2576/00

(57) ABSTRACT

An image processing method for interventional imaging in which a region of interest of a patient is viewed. The method comprises acquiring a succession of images of a region of interest of the patient. The method also comprises detecting and tracking, on the successive images, at least one surgical instrument introduced inside the region of interest of the patient, in order to isolate said instrument therein; and comparing two successive images on which the surgical instrument has been isolated in order to identify at least one common shape therein. The method further comprises estimating the displacement of said common shape between both of these successive images; and re-alignment processing of the different successive images depending on the thereby determined estimations of displacements, these displacement estimations being considered as corresponding to the displacement caused by the physiological movement of the patient with the exception of any other movement.

### CROSS-REFERENCE TO RELATED APPLICATIONS

This application is a continuation of U.S. patent application Ser. No. 12/350,585, filed on Jan. 8, 2009, which claims foreign priority benefits to French Application No. 0850133, filed on Jan. 10, 2008, all of which are incorporated by reference herein in their entireties.

### STATEMENT REGARDING FEDERALLY SPONSORED RESEARCH OR DEVELOPMENT

Not Applicable

### NAMES OF PARTIES TO A JOINT RESEARCH AGREEMENT

Not Applicable

### REFERENCE TO A SEQUENCE LISTING, A TABLE, OR COMPUTER PROGRAM LISTING APPENDIX SUBMITTED ON COMPACT DISC

Not Applicable

### BACKGROUND OF THE INVENTION

1. Field of the invention

The field of the invention relates to medical imaging; and more particularly relates to processing images in interventional radiology (fluoroscopic images).

Additionally the field of the invention relates to a method and a system with which the position of a surgical instrument may be displayed in real time in a region of interest of a patient.

2. Description of Related Art

The principle of interventional radiology for a practitioner consists of guiding and deploying a surgical instrument inside the vascular system of a patient while being assisted by a medical imaging system.

Such a medical imaging system allows the acquisition, processing and real time display of two-dimensional (2D) images representing the vascular system of the patient and the surgical instrument. With these images, the practitioner may guide the instrument in the vascular system.

Acquisition of these images requires the emission of a small dose of X-rays to the patient, images on which the vessels are visible by means of a contrast product injected beforehand into the vascular system of the patient

In order to view the surgical instrument inside the vessels of the patient, a 2D or 3D image of the vascular system of the patient (i.e. a 2D or 3D mask of the mapping of the vascular system of the patient) is acquired (by emitting a small dose of X-rays towards the patient, image on which the vessels are made visible by injection of a contrast product) and is superposed to a 2D image acquired in real time. In this respect, reference may be made to the following scientific Publication.

S. Gorges et al.—3D Augmented Fluoroscopy in Interventional Neuroradiology: Precision Assessment and First Evaluation on Clinical Cases—In Workshop AMI-ARCS 2006 held in conjunction with MICCAI'06, October 2006, Copenhagen, Denmark.

A problem is that considering the fact that two images are superposed, any alignment defect is prejudicial as to the visible result: the practitioner may see the instrument outside the vascular system which is detrimental to the precision required for the procedure of the practitioner.

Such an alignment defect is caused by physiological movement(s) of the patient (breathing for example). These movements complexify the guiding of the instrument since the practitioner has only access to real-time images on which the instrument may appear outside the 2D or 3D mask.

Consequently, a need for taking into account physiological movements of the patient is required in order to improve the duration on the one hand and the quality of the operation on the other hand.

Techniques are known with which physiological movements of a patient may be compensated.

One technique is to use internal or external sensors (see Jochen Krucker, Sheng Xu, Neil Glossop, Anand Viswanathan, Jam Borgert and Bradford J. Wood, Heinrich Schulz—Electromagnetic Tracking for Thermal Ablation and Biopsy Guidance: Clinical Evaluation of Spatial Accuracy Journal of Vascular and Interventional Radiology Volume 18, Issue 9, September 2007, pages 1141-1150).

This technique requires the application of an electromagnetic or optical navigation device which is a clinical limitation.

Another technique is to refer to an internal element of the body of the patient having strong contrast, for example the diaphragm (Alexandre Condurachea, Til Aacha Kai Eckb, Jorg Brednob and Thomas Stehieb—Fast and robust diaphragm detection and tracking in cardiac X-ray projection images—In Proceedings of the SPIE, Volume 5747, pages 17654775, 2005).

Finally, this last technique is not compatible with the dimensions of the X-ray emission field for acquiring fluoroscopic images.

### BRIEF SUMMARY OF THE INVENTION

With embodiments of the invention, it is possible to characterize and to compensate in real time the physiological movement of a patient during an operation by detecting the surgical instrument in the acquired image.

Thus, according to a first aspect, an embodiment of the invention relates to an image processing method for interventional imaging in which a region of interest of a patient is viewed, comprising an acquisition of a succession of images of a region of interest of the patient.

The method further comprises: detecting and tracking, on successive images, at least one surgical instrument introduced inside the region of interest of the patient, in order to isolate said instrument therein; comparing two successive images on which the surgical instrument has been isolated in order to identify at least a common shape therein; estimating the displacement of said common shape between both of these successive images; processing for re-aligning different successive images depending on the thereby determined estimations of displacements, these displacement estimations being considered as corresponding to the displacement caused by the physiological movement of the patient with the exception of any other movement.

In order to detect and track the surgical instrument, operations are applied consisting of applying a mathematical morphological operation on the acquired images; filtering the images on which the mathematical morphological operation has been applied so that each pixel of the images is associated with a certain probability; processing the obtained probabilities in order to make a mapping intended to cause a set of pixels to stand out, representing the instrument.

The estimation processing determines a deformation induced by the movement of the instrument with the exception of any other movement.

Moreover, within the scope of the re-alignment processing, the estimated deformation is applied on a three-dimensional mask of the region of interest of the patient in order to obtain a three-dimensional image on which the physiological movement of the patient is compensated; or on the whole of an image.

Consequently, by means of the re-alignment which only considers the physiological movement, the image delivered to the practitioner is free of alignment defects; the instrument is always inside the mask of the vascular system of the patient.

Further, with an embodiment of the invention, the surgical instrument displaced by the practitioner, set into a relationship with a 2D or 3D mask representing the anatomy of the patient, may be tracked in real time. The operation is improved: it is faster and more efficient.

According to a second aspect, an embodiment of the invention relates to a medical imaging system comprising: means for obtaining an image of a region of interest of a patient; means for acquiring two successive images of the region of interest of the patient.

The system comprises processing means capable of: detecting and tracking on successive images at least one surgical instrument introduced inside the region of interest of the patient, in order to isolate said instrument therein; comparing two successive images on which the surgical instrument has been isolated in order to identify at least one common shape therein; estimating the displacement of said common shape between both of these successive images; processing the realignment of the different successive images depending on the thereby determined estimations of displacements, these displacement estimations being considered as corresponding to the displacement caused by the physiological movement of the patient with the exception of any other movement.

And finally according to a third aspect, an embodiment of the invention relates to a computer program.

The computer program comprises machine instructions for applying a method according to the first aspect of the invention,

### BRIEF DESCRIPTION OF THE DRAWINGS

Other characteristics and advantages of embodiments of the invention will further become apparent from the description which follows, which is purely illustrative and non-limiting and should be read with reference to the appended drawings wherein:

FIG. 1 schematically illustrates a medical imaging system;

FIG. 2 illustrates an image processing method in interventional imaging according to the invention;

FIGS. 3a, 3b and 3c respectively illustrate a vessel of the patient; the vessel comprising an instrument inside it at instant t; the vessel comprising the instrument at instant t+1; and

FIGS. 4a, 4b, 4c and 4d illustrate results obtained by means of the method according to the invention.

### DETAILED DESCRIPTION OF THE INVENTION

### Medical Imaging System

During an interventional radiology operation, a practitioner brings a surgical instrument towards an area to be treated inside the body of the patient by passing through the vascular system of the patient.

The surgical instrument may be a catheter, a guide wire or any other instrument known to one skilled in the art.

In order to facilitate the displacement of the instrument—as already mentioned—with the medical imaging system the instrument inside the vascular system of the patient may be displayed.

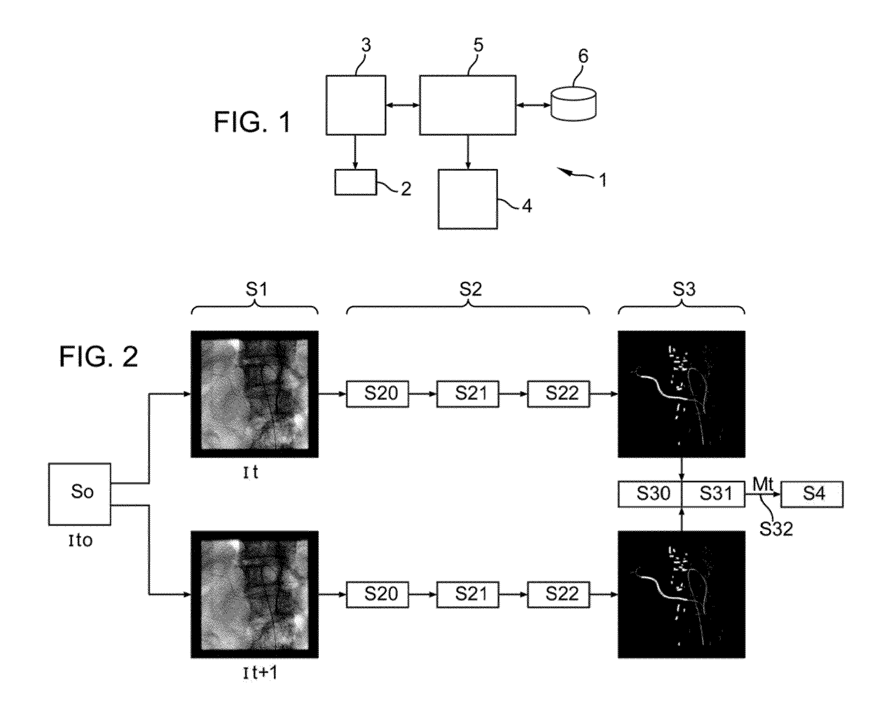

In FIG. 1, the medical imaging system 1 is schematically illustrated, with which a 2D image of an object 2 may be acquired and the acquired 2D image may be processed in order to display the 3D output image for assisting the practitioner with progression of the instrument.

The medical imaging system 1 comprises an image acquisition system 3, an image processing system 5 and a display system 4.

With the acquisition system 3, a 2D image representing the surgical instrument and the vascular system of the patient in two dimensions may be acquired.

The processing system 5 is a computer for example. The processing system 5 is coupled with memory means 6 which may be integrated or separate from the processing system 5. These memory means 6 notably provide storage for the 3D model of the vascular system of the patient. These means may be formed by a hard disk, a diskette, a CDROM.

The image acquisition system 3 is an X-ray acquisition system for example, the latter comprising any known means allowing emission of X rays onto the object 2 and acquisition of resulting images.

General description of the image processing, method

In the following, we consider that the surgical instrument is a catheter.

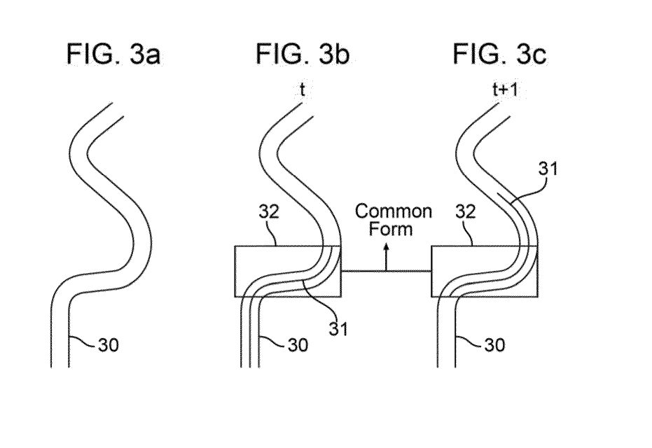

FIG. 2 schematically illustrates the steps of the image processing method provided by an embodiment of the invention. It is considered that the region of interest (the vascular system) of the patient is viewed by means of the medical imaging system.

The method for processing images is based on the following principle.

Step S0: In order to initialize the method, one places oneself at instant t₀ for which no alignment defect is observed in a fluoroscopic 3D image (acquired and reconstructed by means known to one skilled in the art). This initialization may be carried out manually by the practitioner or digitally by means of a computer for example.

Step S1: Two successive images Iₜ, Iₜ₊₁ of a region of interest of the patient are acquired by emitting X-rays on this region by means of the acquisition system 3.

Step S2: During this step, the surgical instrument (catheter, microcatheter, guide wire) IS detected and tracked in the acquired fluoroscopic images Iₜ, Iₜ₊₁.

Step S3: The position of the instrument detected in the image taken at instant t (current instant) is compared (S30) with the position of the instrument detected in the image taken at the preceding instant, instant t-1, in order to estimate a common shape between both images and thus the 2D physiological displacement (S31).

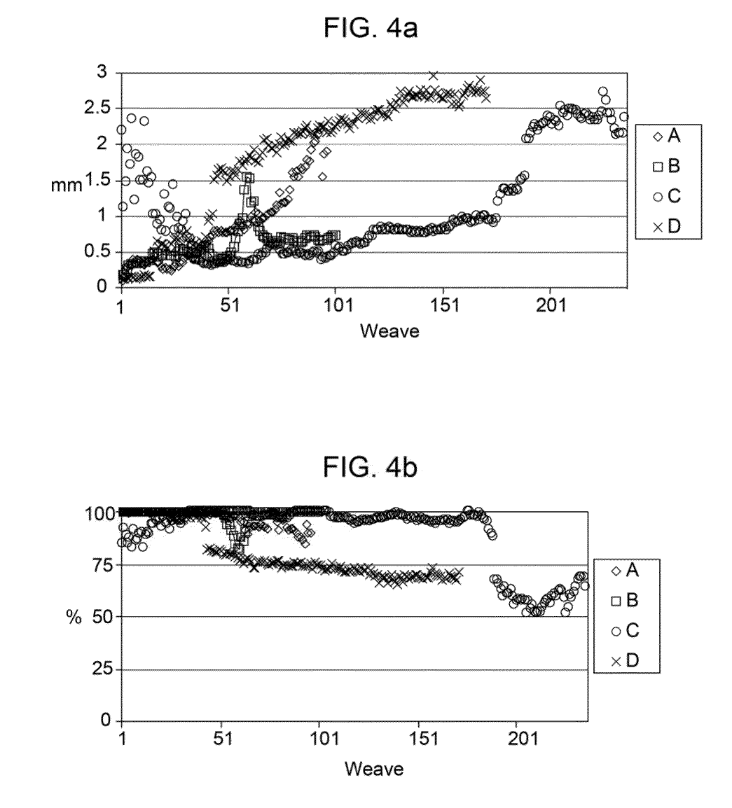

FIGS. 3a, 3b and 3c illustrate what is meant by common shape.

In FIG. 3a, a vessel 30 of the vascular system G illustrated, in which a catheter 30 is introduced (FIGS. 3b and 3c).

FIGS. 3b and 3c correspond to two successive images of the vessel comprising the catheter 31 in two different positions.

The common form 32 which one seeks to estimate between the two successive images is the shape formed by the pair vessel/catheter. In other words, the common portion of the instrument is not sought but it is actually its common shape which is sought.

In FIG. 3c it is seen that the instrument has been subject to a change in length but there is actually a common shape 32 between both images.

It should be noted that the way of estimating the 2D displacement from the displacement of the object depends on the clinical application and on the type of instrument.

From the estimated displacement, the deformation M is determined (S32) between both images.

Step S4: The displacement having been estimated, the inferred deformation M is applied:

either to the complete fluoroscopic image by applying the function M to the image; or

to the 3D (or 2D) mask of the vascular system of the patient by applying the function M to the mask 3D.

As this will have been understood, the method is based on the estimation of the 2D physiological movement by using two images acquired at two successive instants t and t+1.

Detailed description of the steps of the image processing method

The following steps were performed for each of the two images Iₜ and Iₜ₊₁ acquired successively.

Step S2: This step aims at detecting and tracking the movement of the tool in the vascular system of the patient

During a step S20, by a mathematical morphological operation on the acquired images Iₜ and Iₜ₊₁, all the elements of the image other than the instrument are eliminated, for example the elements having a thickness larger than the diameter of a guide wire, in the case when the instrument is a guide wire. A description of the mathematical morphological operations will be found in Jean Serra—Image Analysis and Mathematical Morphology (Vol. 1), Academic Press—London, 1982.

During a step S21, filtering is performed on the thereby obtained image (for example, a filter a so-called “Turning Oriented Filter”, see for example, R. Kutka and S. Stier—Extraction of Line Properties Based on Direction Fields, Transactions on Medical Imaging—Volume 15, p 51-58, February 1996.

Such a filter allows each pixel of the image to be associated with a certain probability of belonging to linear segments having a certain orientation.

And during S22, by a mapping applied to the obtained probabilities, a set of pixels representing the instrument is obtained.

Step S3: The pixels belonging to the instrument detected in each image Iₜ and Iₜ₊₁ are applied here to these same images by using an ICP (Iterative Closest Point) algorithm which is a re-alignment process (S32). A general description of the ICP algorithm may be found in Iterative Point Matching for Registration of Free-Form Curves and Surfaces (1992) (Zhengyou Zhang).

This algorithm iteratively seeks the deformation M (i.e. the transformation) by minimizing a criterion C between two set of points F={(xᵢ,yᵢ)} and V={(wⱼ,zⱼ)}. The criteria to be minimized allows the following expression

C(M) =Σ_{iεI}ρ(||M(x_{i,k}, y_{i,k})−(w_{i,k}z_{i,k})||),

wherein ρ is an estimator of M (see P. J. Huber, “Robust Statistics”, Wiley, New York, 1981) corresponding to the bi-weight function of Tuckey. This function minimizes the influence of interferences.

The algorithm for tracking the tool inside the vascular system of the patient may be summarized in the following way.

The steps below are iterated over the whole duration of the operation.

WHILE (t)

IF t=0 THEN
Let Fₜ be the set of detected pixels in the region of interest of image T₀
Let Vₜ be the set of detected pixels in the region of interest of image I₁

ELSE

Let Vₜ be he set of detected pixels in the region of interest of image Iₜ

END IF

EXECUTE the ICP algorithm in order to estimate the deformation Mₜ which allows passing from Fₜ to Vₜ

Fₜ₊₁=these are the common points of Vₜ selected by the ICP algorithm plus the neighbouring points selecting by the FOT filter.

END WHILE

By means of the estimator of the deformation M, the region of interest may contain detected objects such as agraffes for example, which follow a movement different to that of the instrument. These objects are considered as interfering objects and will not be taken into account in the estimation of the movement.

By means of the ICP algorithm, a change in the length of the guide induced by the practitioner (when the latter notably progresses into the vascular system of the patient) will also not be taken into account in the estimation of the movement.

Indeed, only the common shapes between the images Iₜ and Iₜ₊₁ are taken into account because the sudden changes in length and in shape (initiated by the practitioner) are not taken into account by the bi-weight function of Tuckey.

It should be noted that application of the ICP algorithm may be carried out on a region of interest in order to improve the speed of the processing method.

Step S4: Once the deformation M is estimated, it is applied onto the fluoroscopic image or onto the 2D or 3D mapping of the vascular system of the patient. This latter possibility allows the mask to be displaced, with the breathing movement of the patient visible on the images.

Examples of Results Obtained with the Method Described Above

The method described above was applied to four sequences of fluoroscopic images (noted as A, B, C and D). These sequences were acquired on an Innova4100 C-arm—GE Healthcare system.

The images have dimensions of 1000×1000 and the size of the pixels is 0.2 mm. The length of each sequence is comprised between 150 and 200 images. Each sequence corresponds to a fluoroscopic acquisition on a patient an which a tumour embolization operation is performed.

In these images, only the instrument is visible.

In sequence A, the agraffes are visible: in this example, the patient has been subject to a surgical operation prior to the embolization operation.

Finally, sequences A, B, C and D comprise 3, 1, 6 et 2 breathing cycles, respectively. It is noted that the breathing movement may cause displacement of the instrument as far as 25 mm.

In order to evaluate the accuracy of the re-alignment ICP algorithm, the residual error on the cost function was analyzed. For this purpose, an image recording transformation is applied onto the points of the instrument which have been identified manually at the beginning of the sequence after the filtering operation S11.

Let n be an image and let Fₙ₊₁ be a set of points of the instrument. For each image acquired at instant t, the distance of each point of coordinates (x,y)∈Fₜ₊₁ from the set V_{n . . . t}=M_{n . . . t}(Fₙ₊₁) is determined, where M_{n . . . t}is the transformation which carries out the mapping of the points of the set Vₜ of the pixels detected in the region of interest of the image Iₜ from image n to image t.

This distance corresponds to d=min_{(w,z)∈}V_{n . . . t} ||(w−x)²+(z−y)) ² ||and represents the distance between the instrument in the image t and the instrument in the image n after compensation of the physiological movement.

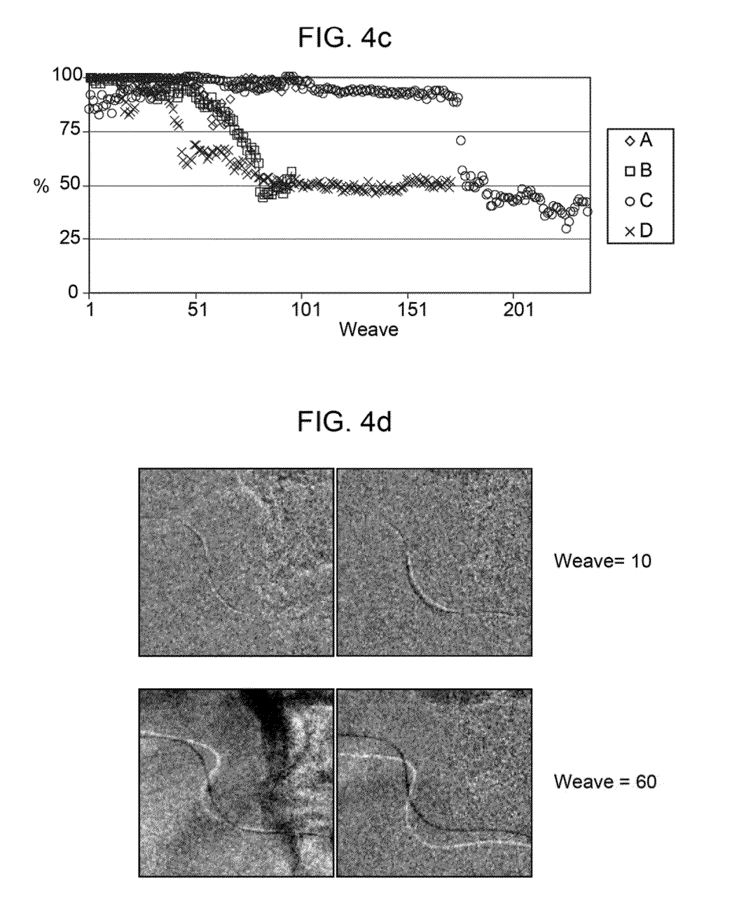

The results are illustrated in FIGS. 4a, 4b, 4c and 4d.

FIG. 4a illustrates the average error of the image recording transformation. It is seen that this error is less than 3 mm for all the sequences and over the whole of their length.

FIGS. 4b and 4c illustrate the percentage of points of the instrument having a tracking error less 3 mm and 6 mm, respectively,

For sequences A and B, the tracking of the installment is accurate: more than 75% of the points have a tracking error less than 3 mm (see FIG. 4c). Moreover, it is seen that for sequence D, the percentage of the points having an error less than 3 mm, changes to 60% around the image of sequence number 50.

Such a phenomenon is explained here by the fact that the movement of the practitioner is not compensated.

FIG. 4d illustrates such a phenomenon. The left figures are the compensated images and the right figures are the non-compensated images for the images numbered 10 and 60. It is observed that as the movement of the patient has been compensated, the instrument is however deformed in the vessels. Indeed, the method does not compensate this movement but this however has the effect of increasing the error of the image recording transformation.

With such a processing method, it is possible to significantly reduce the error due to physiological movements and in particular that induced by the breathing of the patient.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the scope of the following claims.

What is claimed is:

1. A method for processing images for interventional imaging in which a region of interest of a patient is viewed, the method comprising:
acquiring a succession of images of a region of interest of the patient;
detecting and tracking on successive images, at least one surgical instrument introduced inside the region of interest of the patient, in order to isolate said instrument therein; comparing two successive images on which the surgical instrument has been isolated in order to identify at least one common shape therein; estimating the displacement of said common shape between both of these successive images; and
re-alignment processing of the different successive images depending on the thereby determined estimations of displacements, these displacement estimations being considered as corresponding to the displacement caused by the physiological movement of the patient with the exception of any other movement.

2. The method of claim 1, wherein the step of detecting and tracking the surgical instrument, further comprises:
applying a mathematical morphological operation on the acquired images;
filtering the images onto which the mathematical morphological operation has been applied so that each pixel of the images is associated with a certain probability; and
processing the obtained probabilities in order to produce a mapping intended to cause a set of pixels representing the instrument to stand out

3. The method of claim 1, wherein the estimation process determines a deformation induced by the movement of the instrument with the exception of any other movement.

4. The method of claim 3, wherein within the scope of the re-alignment process, the estimated deformation is applied on a three-dimensional mask of the region of interest of the patient so as to obtain a three-dimensional image on which the physiological movement of the patient is compensated.

5. The method of claim 3, wherein within the scope of the re-alignment process, the estimated deformation is applied to the whole of an image.

6. A medical imaging system, comprising:
an imaging device configured to obtain an image of a region of interest of a patient;
an image acquisition device configured to acquire at least two successive images of the region of interest of the patient; and
a processor configured to:
detect and track, on successive images, at least one surgical instrument introduced inside the region of interest of the patient, in order to isolate said instrument therein;
compare two successive images on which the surgical instrument has been isolated for identifying at least one common shape therein;
estimate the displacement of said common shape between both of these successive images; and
process the re-alignment of the different successive images depending on the thereby determined estimations of displacements, these displacement estimations being considered as corresponding to the displacement caused by the physiological movement.
